(19) United States
(12) Patent Application Publication
(10) Pub. No.: US 2022/0366668 A1
(43) Pub. Date: Nov. 17, 2022

(54) IMAGE PROCESSING APPARATUS, IMAGE PROCESSING METHOD, AND IMAGE PROCESSING PROGRAM

(71) Applicant: SONY GROUP CORPORATION, TOKYO (JP)

(72) Inventors: ATSUSHI ITO, TOKYO (JP); TETSU OGAWA, KANAGAWA (JP); KENICHIRO NAKAMURA, TOKYO (JP); YUSUKE MORIUCHI, TOKYO (JP)

(21) Appl. No.: 17/755,153

(22) PCT Filed: Sep. 4, 2020
(86) PCT No.: PCT/JP2020/033523

§ 371 Date: Apr. 22, 2022

(30) Foreign Application Priority Data
Oct. 30, 2019 (JP) 2019-197849

(51) Int. Cl. G06V 10/60; G06V 10/143; H04N 5/247; H04N 9/04; G06V 20/10; G06V 20/17; G06V 10/147; G01N 21/3563; G01N 21/359; G01N 21/84; G01N 33/00

(52) U.S. Cl. CPC G06V 10/60; G06V 10/143; H04N 5/247; H04N 9/0455; G06V 20/188; G06V 20/17; G06V 10/147; G01N 21/3563; G01N 21/359; G01N 21/84; G01N 33/0098; G01N 2021/8466

(57) ABSTRACT

Provided are an image processing apparatus, an image processing method, and an image processing program capable of achieving high accuracy in an index representing vegetation. An image processing apparatus (1) includes a normal map generation unit (12) and a reflection characteristic model generation unit (18). The normal map generation unit (12) obtains a normal vector characteristic based on a polarized image acquired. The reflection characteristic model generation unit (18) estimates a reflection characteristic model based on the normal vector characteristic obtained by the normal map generation unit (12).

### FIELD

The present disclosure relates to an image processing apparatus, an image processing method, and an image processing program.

### BACKGROUND

Remote sensing is a technology of measuring a target from a remote distance over a wide area. In the agricultural domain, remote sensing is often used for the purpose of measuring plant functions from artificial satellites and the like. In recent years, in order to implement measurement with higher spatial resolution, there has been remarkable development in the measurement using unmanned aerial vehicles (UAVs) represented by drones.

Among remote sensing technologies, a technology referred to as reflection spectroscopic remote sensing is often used to measure plant functions. The reflection spectroscopic remote sensing includes spectroscopic observation of reflected light from plants with visible to near-infrared wavelengths (400 mm to 2500 mm) using a multispectral camera or a hyperspectral camera. The observed spectroscopic data is used to estimate information such as an internal structure of a plant, the type and amount of pigments and trace components contained, and the water state.

### CITATION LIST

### Patent Literature

Patent Literature 1: WO 2012/073519 A

### SUMMARY

### Technical Problem

In reflection spectroscopic remote sensing, however, there is a concern of occurrence of large variation in measured values depending on an observation environment. The observation environment includes irradiation-related factors such as states of clouds and the color temperature and angle of the sun as well as the geometrical relationship between the angle of the remote sensing imaging system and the target field surface. Occurrence of variation in the measured values due to the observation environment in this manner would make it difficult to achieve high accuracy in an index representing vegetation calculated using the observation data.

In view of this, the present disclosure provides an image processing apparatus, an image processing method, and an image processing program capable of achieving high accuracy in an index representing vegetation.

### Solution to Problem

According to the present disclosure, an image processing apparatus includes: a vector analysis unit that obtains a normal vector characteristic based on a polarized image acquired; and a characteristic estimation unit that estimates a reflection characteristic model based on the normal vector characteristic obtained by the vector analysis unit.

### BRIEF DESCRIPTION OF DRAWINGS

FIG. 1 is a diagram illustrating an image processing system according to a first embodiment.

FIG. 2 is a block diagram of an image processing apparatus.

FIG. 3 is a diagram illustrating a concept of NDVI.

FIG. 4 is a diagram illustrating reflection characteristics of vegetation.

FIG. 5 is a diagram illustrating reflected light incident on a drone.

FIG. 6 is a diagram illustrating a typical problem caused by reflection characteristics.

FIG. 7 is a diagram illustrating an outline of the explanation of each parameter of a PROSAIL model.

FIG. 8 is a diagram illustrating a table summarizing the explanation of each parameter of a PROSAIL model.

FIG. 9 is a diagram illustrating an example of a processing result of the region division processing.

FIG. 10 is a diagram illustrating an example of a result of acquiring a normal map from a polarized image of vegetation.

FIG. 11 is a diagram illustrating a mathematical representation of LIDF and a measurement histogram which is a descriptive distribution thereof.

FIG. 12 is a diagram illustrating a result of leaf detection using a reflection spectroscopic image.

FIG. 13 is a diagram illustrating a result of leaf detection using a polarized image.

FIG. 14 is a flowchart of a reflection characteristic model generation process.

FIG. 15 is a diagram illustrating an example of an imaging device according to a second embodiment.

FIG. 16 is a diagram illustrating an example of an imaging device according to a modification of the second embodiment.

FIG. 17 is a diagram illustrating acquisition of a narrowband R/IR signal by a combination of a bandpass filter and an RGB sensor.

FIG. 18 is a diagram illustrating an example of an imaging device according to a third embodiment.

FIG. 19 is a diagram illustrating another example of an imaging device according to the third embodiment.

FIG. 20 is a diagram illustrating an example of an imaging device according to a fourth embodiment.

### DESCRIPTION OF EMBODIMENTS

Embodiments of the present disclosure will be described below in detail with reference to the drawings. In each of the following embodiments, the same parts are denoted by the same reference symbols, and a repetitive description thereof will be omitted.

### First Embodiment

[Configuration of System According to First Embodiment]

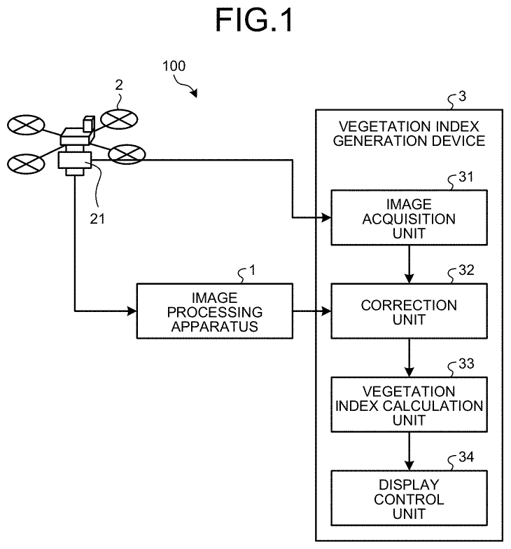

FIG. 1 is a diagram illustrating an image processing system according to a first embodiment. As illustrated in FIG. 1, an image processing system 100 includes an image processing apparatus 1, a drone 2, and a vegetation index generation device 3.

The image processing system 100 is a system that provides a vegetation index for estimating vegetation information, which is information related to the distribution, amount, and function of vegetation at implementation of sensing referred to as reflection spectroscopic remote sensing.

The drone 2 is equipped with an imaging device 21 including a camera that captures a reflection spectroscopic image and a polarized image. Here, the camera that captures the reflection spectroscopic image and the camera that captures the polarized image included in the imaging device 21 may be separate cameras or one camera. For example, the imaging device 21 is equipped with a camera using an imaging element capable of simultaneously acquiring four polarized beams of light at four polarization angles of 0 degrees, 45 degrees, 90 degrees, and 135 degrees in an imaging system standard in which a specific position is defined as 0 degrees among the polarization angles.

The drone 2 flies over a field as a vegetation survey target, and simultaneously acquires a reflection spectroscopic image and a polarized image of the field from an aerial viewpoint using the camera. Thereafter, the drone 2 continuously captures reflection spectroscopic images and polarized images while moving over the field, and connects a series of images to each other to acquire a reflection spectroscopic image group and a polarized image group, which will respectively be reflection spectroscopic images or polarized reflection images covering a part or the whole of the field.

The image processing apparatus 1 is an information processing device that executes image processing according to the present disclosure. The image processing apparatus 1 acquires a polarized image group from the drone 2. Here, the image processing apparatus 1 is connected to the drone 2 with a wireless or wired channel to acquire the data of the polarized image group. The polarized image group includes a plurality of polarized images for each polarization angle. However, image processing of one polarized image will be described below since the image processing apparatus 1 performs similar processing on each of the polarized images.

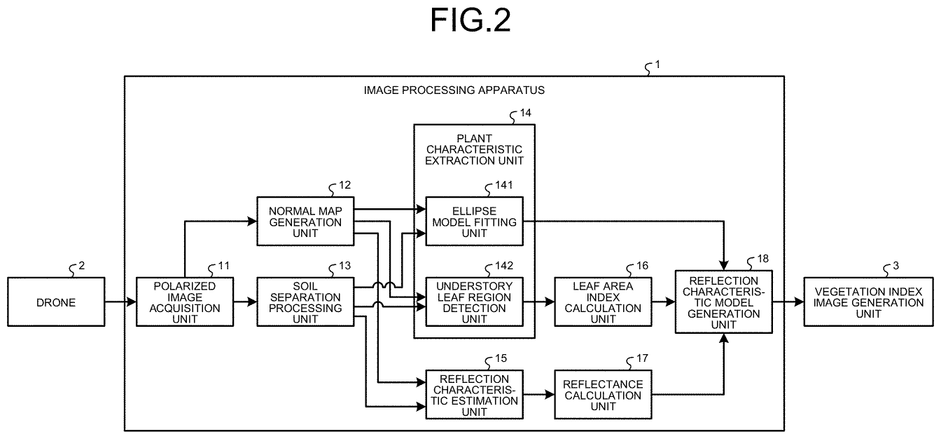

The image processing apparatus 1 acquires a normal vector in each pixel from the polarized image. The image processing apparatus 1 then acquires a parameter in a predetermined mathematical model when a normal vector characteristic, which is distribution of the normal vector in the polarized image, is expressed by the predetermined mathematical model. Next, the image processing apparatus 1 estimates a reflection characteristic model representing the intensity of the reflected light in each direction at each point of the vegetation community represented by the polarized image using the obtained parameter. Thereafter, the image processing apparatus 1 outputs the information regarding the estimated reflection characteristic model to the vegetation index generation device 3. FIG. 2 is a block diagram of the image processing apparatus.

Here, reflection spectroscopic remote sensing will be described. By using reflection spectroscopic remote sensing, it is possible to estimate plant information such as an internal structure of the plant, pigments contained in the plant, the type and amount of trace components, and the water state. Examples of estimated contained pigments include chlorophyll a, chlorophyll b and carotenoids. In addition, examples of the estimated trace components include nitrogen, potassium, and phosphorus.

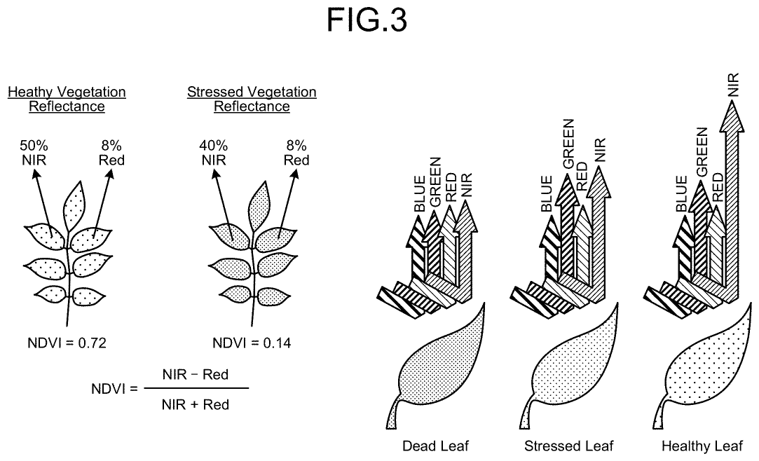

Plant information is often estimated using a formula that uses, as input, reflectance spectroscopic results for the wavelength ranges that are highly correlated with the information as a measurement target. Estimation using this formula sometimes uses an index referred to as Vegetation Index (VI). The most typical VI is the Normalized Difference Vegetation index (NDVI), which is a normalized ratio of red light of 650 nm or less, and near infrared (NIR) light of 800 nm or less illustrated in FIG. 3. FIG. 3 is a diagram illustrating a concept of NDVI. In the following, near-infrared light may be simply referred to as NIR. NDVI is an index that roughly indicates the health of plants by utilizing the absorption of chlorophyll pigment in the red band and the high reflection characteristics of the cell structure of plants in the NIR band. For example, as illustrated in FIG. 3, NDVI can be used to determine the health of a plant.

Other examples of VI include Normalized Difference Red Edge (NDRE), Normalized Difference Water Index (NDWI), and Normalized Difference Built-up Index (NDBI). NDRE is an index that quantifies the correlation between light in the 710 nm to 720 nm band referred to as a Red Edge and the content of Chlorophyll. NDWI is a normalized ratio of light with a wavelength of 860 nm and light with a wavelength of 1240 nm, which have a high correlation with the water content. NDBI is a normalized ratio of light with a wavelength of 860 nm and light with a wavelength of 2160 nm, which are highly correlated with the dry product content.

Here, in the reflection spectroscopic remote sensing, the measured value can greatly vary depending on the geometrical relationship between the illumination/imaging system and the field surface. This is due to the fact that a vegetation community is characteristic compared to other natural objects in terms of the characteristics of the intensity of reflected light depending on the illumination accuracy and the observation angle. In the following, the characteristic of the intensity of reflected light depending on the illumination accuracy and the observation angle may be simply referred to as “reflection characteristic”.

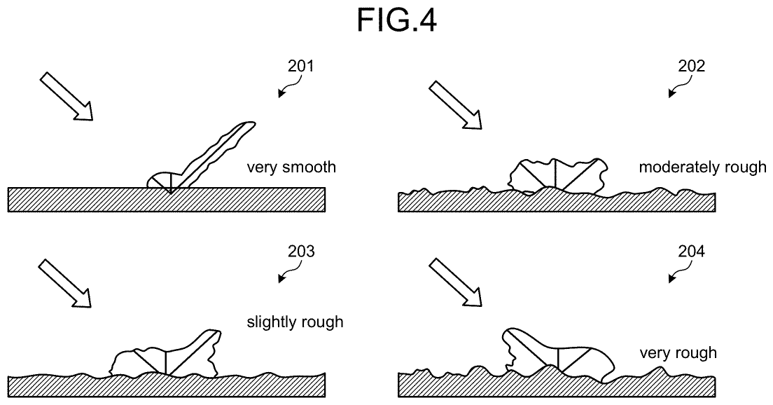

FIG. 4 is a diagram illustrating reflection characteristics of vegetation. On an assumption that light from a light source is incident from directions of arrows, the region surrounded by the curve illustrated on a reflecting surface represents the intensity of the reflected light observed in the individual directions. Normally, on a reflecting surface formed of a flat and smooth material, strong light is reflected on the opposite side of the reflecting surface across the normal for the incident angle of the illumination light from the light source, as illustrated in a reflection characteristic 201. In contrast, when a vegetation community is a target, the reflecting surface is often formed of rough material. In the case of a reflective surface formed of a rough material, as illustrated in a reflection characteristic 204, the reflection intensity in the same direction as the direction in which the light source is located is typically stronger with respect to the normal of the reflecting surface due to the influence of internal interreflection and the like. In addition, as illustrated in FIG. 4, depending on the state of the reflecting surface, there can be occurrence of reflection having a reflection characteristic 202 or 203. Hereinafter, models demonstrating the intensity of the reflected light in individual observation directions according to the reflection characteristics, which are illustrated in the reflection characteristics 201 to 204, are referred to as “reflection characteristic models”.

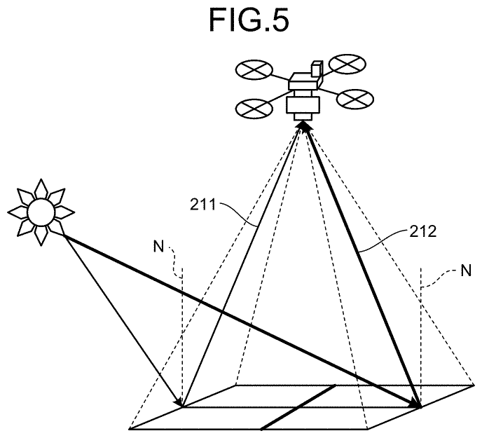

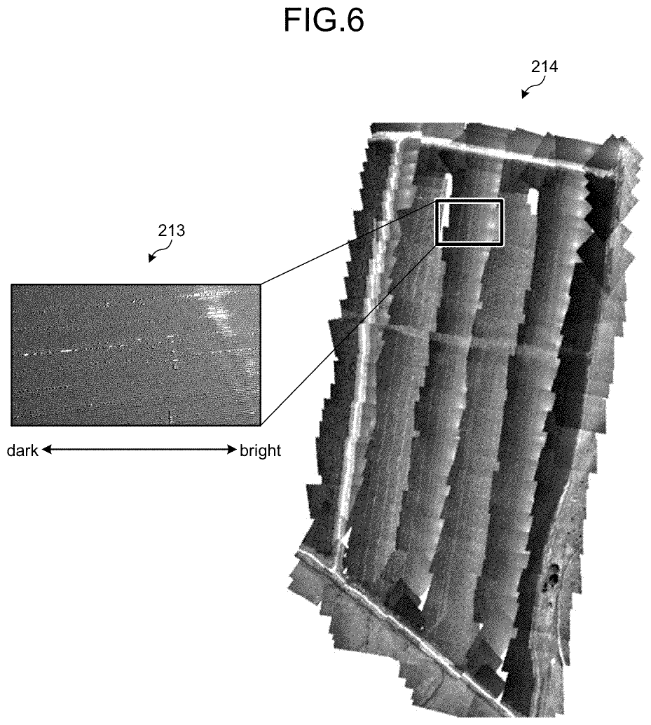

FIG. 5 is a diagram illustrating the reflected light incident on a drone. FIG. 6 is a diagram illustrating a typical problem caused by reflection characteristics. As illustrated in FIG. 5, when a ground directly below a drone is imaged from the drone, reflected light 211 at the left end of the image and reflected light 212 at the right end of the image have a positive/negative relationship across a normal N in observation angles with respect to the sun angle. At this time, in a case where the plant community has the reflection characteristic 204 in FIG. 4, the observed image has a difference in luminance between the right end and the left end as illustrated in a captured image 213 of FIG. 6. In the captured image 213, luminance is low in the left end and high in the right end, when viewed on the page. Combining the captured image 213 like this together as the entire field without correction would generate an image 214. In this case, when there is a large difference in the level of luminance, there will be an image having a state in which a dark region and a bright region are alternately drawn as illustrated in the image 214. The image 214 in such a state is considered to be far from an image matching the state of the actual plant community. In this manner, an occurrence of variation in the measured values depending on the observation conditions can lead to a big problem in the reflection spectroscopic remote sensing.

As a technique of correcting such variation in the measured values due to the reflection characteristics, there is a method of estimating the reflection characteristics of the vegetation community in the field as a measurement target and correcting the measured values accordingly. For example, in order to add correction according to the reflection characteristic to the measured value of each pixel in image measurement, it is conceivable to express the reflection characteristic by a mathematical model. In that case, the model parameters of the mathematical model are estimated.

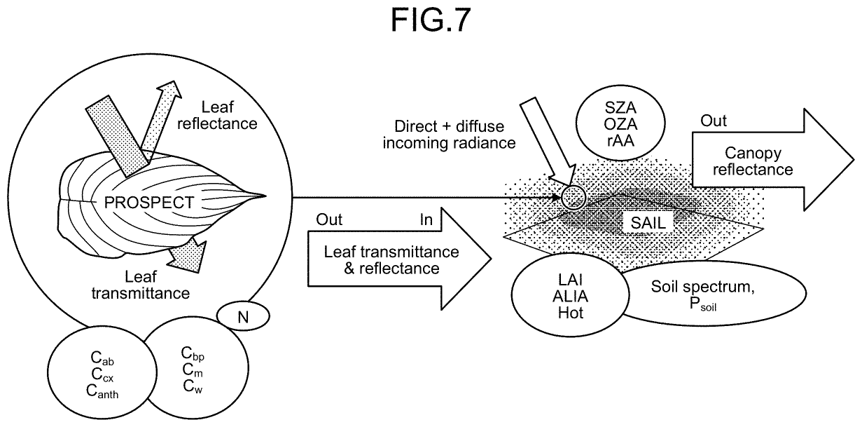

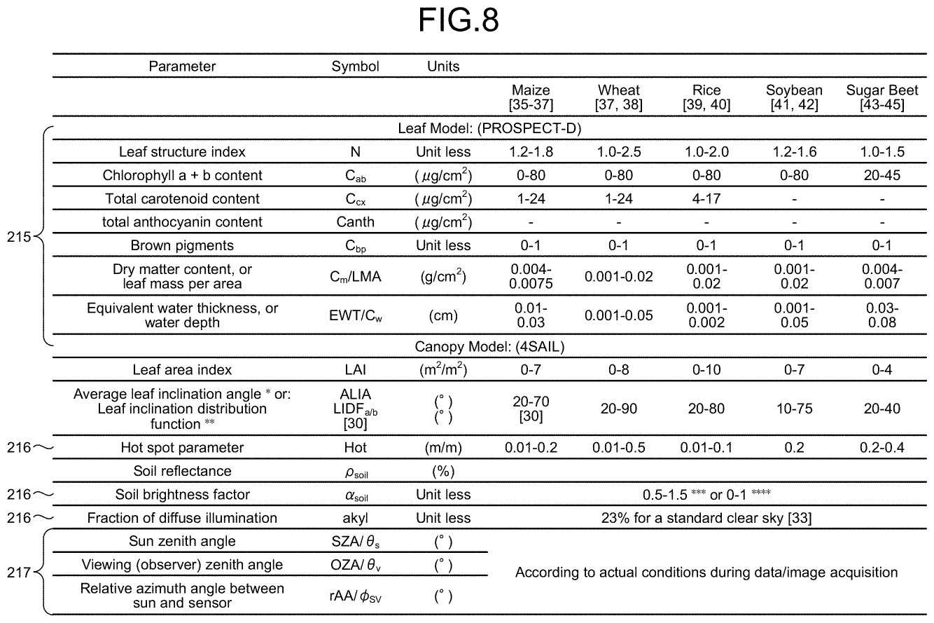

One of the most frequently used mathematical models is a mathematical model referred to as PROSAIL. For example, “Katja Berger, Clement Atzberger, Martin Danner, Guid D′Urso, Wolfram Mauser, Francesco Vuolo, Tobias Hank, Evaluation of the PROSAIL Model Capabilities for Future Hyperspectral Model Environments: A Review Study, Remote Sensing (MDPI), Jan. 10, 2018.” describes a PROSAIL model that exhibits a reflection characteristic of a vegetation community. FIG. 7 is a diagram illustrating an outline of the explanation of each parameter of a PROSAIL model. FIG. 8 is a diagram illustrating a table summarizing the explanation of each parameter of a PROSAIL model. From FIGS. 7 and 8, it can be seen that the parameters for describing the PROSAIL model include parameters related to the sun direction, observation direction and atmosphere, as well as parameters related to vegetation type, community status and soil status. The former parameters can be fixed to be constant in the observation environment. On the other hand, the latter parameters are field-specific values, and it is difficult to accurately specify each of them.

An example of parameters related to vegetation type, community status and soil status is leaf normal statistics. For example, Average Leaf Inclination Angle (ALIA) and Leaf Inclination Distribution Function (LIDF) in FIG. 8 are examples of leaf normal statistics. ALIA is an average value of values that indicate an inclination angle of the leaf with respect to the zenith. LIDF is a value that expresses the distribution of inclination angles with respect to the zenith. As illustrated in “G. S Campbell, Derivation of an angle density function for canopies with ellipsoidal leaf angle distributions, Agricultural and Forest Meteorology, Volume 49, Feb. 3, 1990, 173-176.”, LIDF represents the flatness of an ellipsoid that approximates the normal angle distribution with respect to the zenith of each of leaves in a vegetation community.

In general, the PROSAIL model is effective for describing the reflection characteristic of the vegetation community and is widely used for analysis of sensing results from the sky, such as satellite observations. However, there are many parameters used for the analysis, and handling of these parameters is complicated. Above all, it is difficult to derive field-specific parameters ALIA and LIDF from remote sensing information.

Therefore, there is a proposed technique in which information measured separately on the ground as field-related data is used as auxiliary information for remote sensing to supplement the accuracy of derivation of the parameters of ALIA and LIF. However, such measurements often use special sampling methods and in that case, there is a concern that the cost will increase. In addition, a measurement of field-related data performed separately on the ground has a limited range of the measurement, and thus tend to use only sampled measurement results in the processing. This is likely to cause limited guarantee of accuracy for a wide range of targets, making it difficult to improve the accuracy of measured values in an appropriate range.

In view of these issues, the image processing apparatus 1 according to the present disclosure analyzes normal vector characteristics from a polarized image, acquires normal vector characteristic parameters representing a mathematical model exhibiting normal vector characteristics, and estimates a reflection characteristic model for a vegetation community using the acquired normal vector characteristic parameters. This makes it possible for the image processing apparatus 1 according to the present disclosure to perform easy and high-accuracy acquisition of a reflection characteristic model of the vegetation community, correction of variations attributed to the reflection characteristics regarding the index value representing vegetation calculated from the observation data from the sky, leading to the acquisition of an index representing accurate vegetation. Hereinafter, the image processing apparatus 1 according to the present disclosure will be described in detail with reference to FIG. 2.

The image processing apparatus 1 includes a polarized image acquisition unit 11, a normal map generation unit 12, a soil separation processing unit 13, a plant characteristic extraction unit 14, a reflection characteristic estimation unit 15, a leaf area index calculation unit 16, a reflectance calculation unit 17, and a reflection characteristic model generation unit 18. The plant characteristic extraction unit 14 has an ellipse model fitting unit 141 and an understory leaf region detection unit 142.

The polarized image acquisition unit 11 acquires a polarized image captured by the drone 2. Next, the polarized image acquisition unit 11 outputs the acquired polarized image to the normal map generation unit 12 and the soil separation processing unit 13.

The normal map generation unit 12 receives input of the polarized image from the polarized image acquisition unit 11. Next, the normal map generation unit 12 performs image processing on the polarized image and detects the normal of a leaf on a pixel-by-pixel basis. Subsequently, the normal map generation unit 12 generates a normal map representing the distribution of the normals.

For example, the normal map generation unit 12 calculates normal information by applying polarized images in a plurality of directions to a model formula to generate a normal map. This normal map is an example of a “normal vector characteristic”. More specifically, the normal map generation unit 12 obtains the azimuth from the phase of the observed light when the observed luminance is applied to the following Mathematical Formula (1).

$$I = \frac{{I}_{max} + {I}_{min}}{2} + \frac{{I}_{max} - {I}_{min}}{2} cos ( 2 {θ}_{pol} - 2 Φ ) \qquad ( 1 )$$

Here, I is observed luminance through a polarizing plate. θₚₒₗ is an angle of the rotated polarizing plate. ϕ is a phase of the observed light. Iₘₐₓ and Iₘᵢₙ are amplitudes of fitting.

Furthermore, the normal map generation unit 12 obtains a zenith angle by using the equation of the degrees of polarization represented by the following Mathematical Formula (2). The degrees of polarization represents the ratio of polarized light in the observed light, and generally, the degrees of polarization increases with an increase in the zenith angle.

$$ρ = \frac{{I}_{max} - {I}_{min}}{{I}_{max} + {I}_{min}} \qquad ( 2 )$$

Examples of the technique of calculating the normal map include Japanese Patent Application Laid-Open No. 2007-86720, International Publication No. 2008/099589, and Lawrence B. Wolff et.al., Constraining Object Features Using Polarization Reflectance Model, 1991, Gary A. Atkinson et.al., Recovery of Surface Orientation From Diffuse Polarization, 2006.

The process of obtaining a normal map does not particularly limit the wavelength of polarization. Furthermore, like the normal map generation unit 12 according to the present embodiment, it is possible to improve the accuracy of the normal map by using RGB color information together with the polarization information. For example, Miyazaki Daisuke et al. Polarization-based inverse Rendering from a single view. ICCV03 (9820987) discloses a method of solving the normal accuracy deterioration due to the difference in polarization behavior between the specular reflection and diffuse reflection. Specifically, the disclosed technique is a technique of improving the normal estimation accuracy by achieving match between the polarization behavior and the diffuse reflection behavior by executing pre-signal processing of removing the specular reflection component using the individual color and polarization information. The normal map generation unit 12 can also obtain a similar effect by using RGB color information together with polarization information.

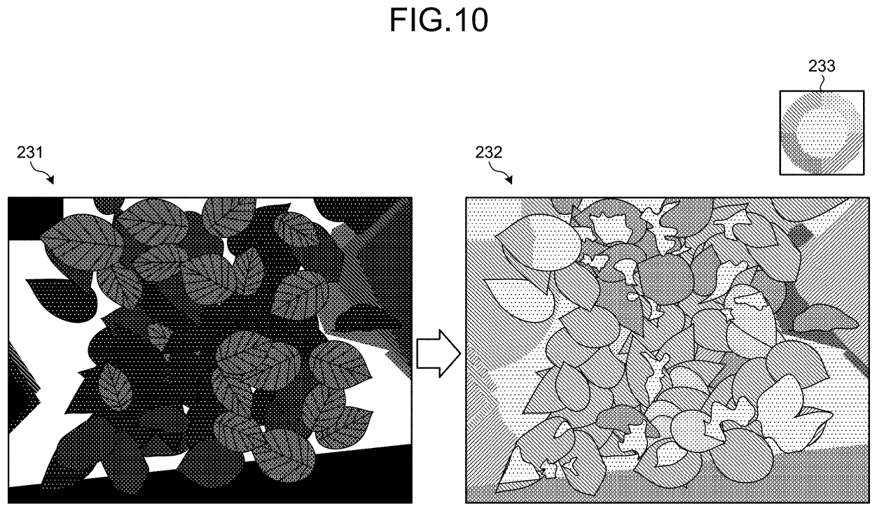

FIG. 10 is a diagram illustrating an example of a result of acquiring a normal map from a polarized image of vegetation. A polarized image 231 is a polarized image in the 0 degrees direction in the imaging system standard. For example, the normal map generation unit 12 generates a normal map 232 from the polarized image 231. The normal map 232 illustrates the direction of the normal for each pixel. The normal map 233 is a normal map regarding a reference sphere.

The normal map generation unit 12 outputs the generated normal map to the ellipse model fitting unit 141, the understory leaf region detection unit 142, and the reflection characteristic estimation unit 15. This normal map generation unit 12 corresponds to an example of a “vector analysis unit”.

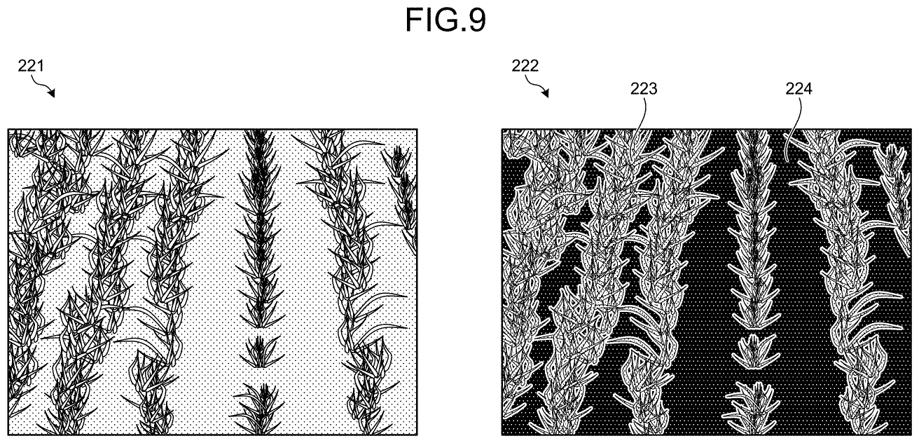

The soil separation processing unit 13 receives input of the polarized image from the polarized image acquisition unit 11. Subsequently, by image processing, the soil separation processing unit 13 executes a region division processing of dividing a polarized image into a vegetation region and a soil region. The details of the region division processing will be described below. FIG. 9 is a diagram illustrating an example of a processing result of the region division processing.

The soil separation processing unit 13 divides the region into the vegetation region and the soil region in the polarized image by using a general color segmentation technique. In addition, the soil separation processing unit 13 performs this region division processing on each of the polarized images included in the polarized image group so as to improve the separation accuracy. For example, by performing the region division processing on a polarized image 221 of FIG. 9, the soil separation processing unit 13 acquires a division processed image 222. In the division processed image 222, a region 223 is a vegetation region while a region 224 is a soil region.

Subsequently, the soil separation processing unit 13 outputs a signal of a region determined to be the soil region in the polarized image to the reflection characteristic estimation unit 15. Furthermore, the soil separation processing unit 13 outputs a signal of a region determined to be a vegetation region in the polarized image to the ellipse model fitting unit 141 and the understory leaf region detection unit 142 of the plant characteristic extraction unit 14.

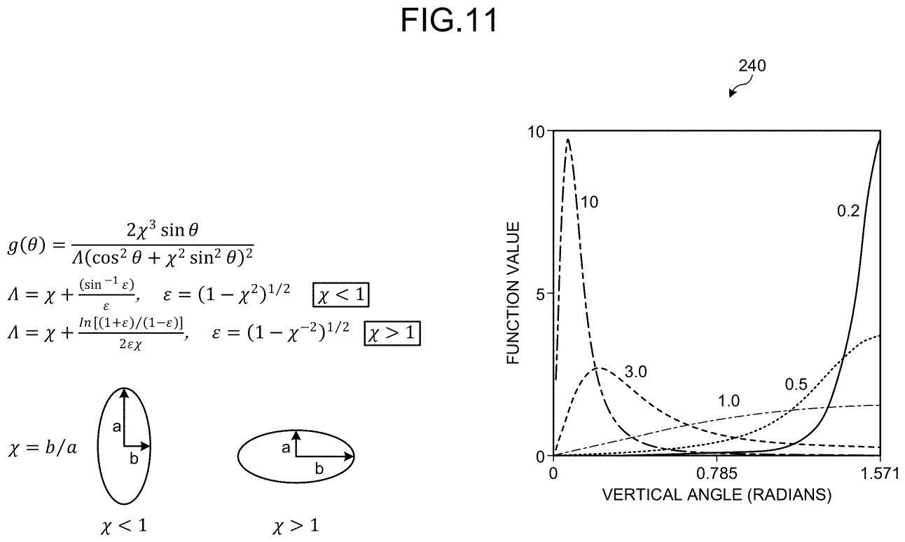

The ellipse model fitting unit 141 receives input of the normal map from the normal map generation unit 12. Furthermore, the ellipse model fitting unit 141 receives input of the signal of the image of the vegetation region in the polarized image from the soil separation processing unit 13. The ellipse model fitting unit 141 specifies a region corresponding to the vegetation region of the normal map. Subsequently, by using the normal distribution illustrated in FIG. 11 on the normal information in the specified region, the ellipse model fitting unit 141 obtains the optimum parameters for a mathematical approximation model using the ellipse. FIG. 11 is a diagram illustrating a mathematical representation of LIDF and a measurement histogram which is a descriptive distribution of the representation. Specifically, the ellipse model fitting unit 141 obtains χ in FIG. 11 as a parameter. The χ obtained by the ellipse model fitting unit 141 corresponds to the LIDF of the mathematical approximation model using the ellipse.

An intermediate output value Λ corresponding to the value of χ, which is a ratio of a major axis and a minor axis of an ellipse, is calculated from the mathematical representation of LIDF in FIG. 11, and then, g(θ) representing the zenith angle distribution is finally calculated. The curve expressed by the mathematical representation is illustrated by a histogram 240. In the histogram 240, the vertical axis represents the frequency of normals, and the horizontal axis represents the angle of normals (radians). The closer the angle of normal is to 0, the more horizontally the leaves extend with respect to the ground, and the closer the angle of normal is to 1.571, the more perpendicular the leaves extend with respect to the ground.

The ellipse model fitting unit 141 acquires a depth map by performing three-dimensional sensing such as Light Detection and Ranging (LiDAR) using a normal map and a polarized image of a vegetation region. Thereafter, the ellipse model fitting unit 141 selects χ having a distribution represented by g(θ) that fits the obtained depth map. In this manner, the ellipse model fitting unit 141 performs LIDF parameter fitting for determining the value of the ratio χ. Examples of the methods of searching for χ that fits the depth map include a method of finding χ when there is highest similarity to the measurement histogram by using a general full search, a hill climbing technique, and the like. The ellipse model fitting unit 141 outputs the obtained LIDF to the reflection characteristic model generation unit 18. The LIDF obtained by the ellipse model fitting unit 141 corresponds to an example of “a parameter representing normal distribution”.

The reflection characteristic estimation unit 15 receives input of the signal of the image of the soil region in the polarized image from the soil separation processing unit 13. By receiving the signals of the soil region for each of polarized images included in the polarized image group from the soil separation processing unit 13, the reflection characteristic estimation unit 15 accumulates pixel data obtained by imaging the soil region from various angles. Furthermore, the reflection characteristic estimation unit 15 receives input of the normal map from the normal map generation unit 12.

Next, the reflection characteristic estimation unit 15 determines whether the reflection characteristic of the soil region can be regarded as Lambertian reflection by using the accumulated image data and the normal map. Lambertian reflection is a reflection model that regards a diffuse reflection surface as an ideal surface, in which the reflected light has uniform intensity in all directions. Here, when all the soil regions in the polarized image can be regarded as flat, the reflection characteristic estimation unit 15 can also acquire the reflection characteristics of the soil region without using the normal map.

When the reflection characteristic of the soil region cannot be regarded as the Lambertian reflection, that is, when the reflection characteristic of the soil region is the non-Lambertian reflection, the reflection characteristic estimation unit 15 obtains a mathematical model representing the reflection characteristic. For example, the reflection characteristic estimation unit 15 applies a Bidirectional Reflectance Distribution Function (BRDF) model such as Phong reflection model to the reflection characteristic of the soil region. With this application, the reflection characteristic estimation unit 15 estimates the most approximate parameter in the mathematical model representing the reflected light in the soil region, and obtains a mathematical model representing the reflection characteristic of the soil region. Here, the reflection of the soil region expressed by the reflection model of the Lambertian reflection or the BRDF model is referred to as a “soil reflection characteristic”.

The reflection characteristic estimation unit 15 outputs the estimated soil reflection characteristic to the reflectance calculation unit 17. Here, while the present embodiment is a case where the polarized image is divided into two regions, namely, the vegetation region and the soil region, even when the reflection characteristic estimation unit 15 executes the division processing without completely dividing the polarized image into the two regions while having a certain ambiguous region left, the reflectance can be calculated by the reflectance calculation unit 17 as described below.

The reflectance calculation unit 17 receives input of the estimated soil reflection characteristic from the reflection characteristic estimation unit 15. Subsequently, the reflectance calculation unit 17 calculates a reflectance ρs of the soil using the acquired soil reflection characteristic. Specifically, when the soil has non-Lambertian reflection, the reflectance calculation unit 17 calculates the reflectance ρs by one of the following methods, for example. In one method, the reflectance calculation unit 17 obtains the reflectance ρs by adopting the reflection in a region where the specular reflection is the least. In another method, the reflectance calculation unit 17 performs a computational cancellation of specular reflection and extracts the most stable spectral reflectance as the reflectance ρs.

Thereafter, the reflectance calculation unit 17 outputs the calculated reflectance ρs of the soil to the reflection characteristic model generation unit 18.

In addition, in the PROSAIL model, the contribution of a parameter referred to as the leaf area index (LAI) is also large. The leaf area index is a value obtained by integrating all the leaf areas above a certain land and converting the integrated value into a value per unit land area. Generally proposed methods includes a technique of obtaining the leaf area index by observing the fallen leaves of the target vegetation and a technique of improving the accuracy of the leaf area index by complementation using the light amount difference information compared to the observation on the vegetation community by imaging the community from below. However, these techniques have a limited range of the measurement, and thus tend to use only sampled measurement results in the processing. This is likely to cause limited guarantee of accuracy for a wide range of targets, making it difficult to improve the accuracy of measured values in an appropriate range. Therefore, the image processing apparatus 1 according to the present embodiment obtains the leaf area index using a polarized image and a normal map. The calculation of the leaf area index by the understory leaf region detection unit 142 and the leaf area index calculation unit 16 will be described below.

The understory leaf region detection unit 142 receives input of the normal map from the normal map generation unit 12. Furthermore, the understory leaf region detection unit 142 receives input of the signal of the image of the vegetation region in the polarized image from the soil separation processing unit 13. Subsequently, the understory leaf region detection unit 142 performs edge detection and machine learning using the signal of the image of the vegetation region and the normal map, and estimates the number of leaves in the polarized image.

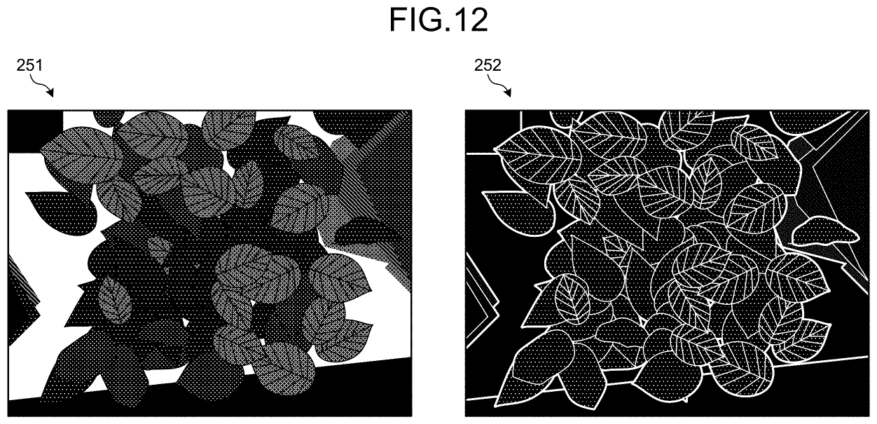

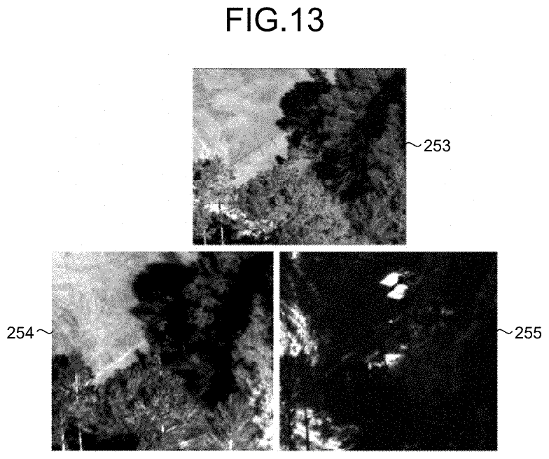

FIG. 12 is a diagram illustrating a result of leaf detection using a reflection spectroscopic image. In addition, FIG. 13 is a diagram illustrating a result of leaf detection using a polarized image. By performing edge detection on a reflection spectroscopic image 251 in FIG. 12, an edge detection image 252 can be obtained. As represented by the edge detection image 252, it is difficult to detect the presence of leaves in shadow regions in the image of the reflection spectroscopic image 251. On the other hand, the understory leaf region detection unit 142 can detect the leaves existing in shadow regions by polarized light in one direction or a combination of beams of polarized light in several polarization directions. A polarized image 254 of FIG. 13 is a polarized image with unidirectional polarization corresponding to a reflection spectroscopic image 253. A polarized image 255 is an image obtained by combining images in several polarization directions. That is, by using the polarized image 254 and the polarized image 255, the understory leaf region detection unit 142 can detect the leaves in the shadow portions that cannot be detected by the reflection spectroscopic image 253. In this manner, the understory leaf region detection unit 142 performs leaf detection on images under a plurality of polarization conditions. The understory leaf region detection unit 142 then calculates the leaf area density from the detected number of leaves. Thereafter, the understory region detection unit 142 outputs the leaf area density to the leaf area index calculation unit 16. In this manner, by detecting the leaves in the shadow region, it is possible to improve the accuracy of the leaf area density, leading to the improvement of the accuracy of the leaf area index calculated by the leaf area index calculation unit 16 in the subsequent process.

Here, although the present embodiment uses a method in which the understory leaf region detection unit 142 calculates the leaf area density using the signal of the region determined to be the vegetation region and using the normal map, the understory leaf region detection unit 142 may obtain the leaf area density without using the normal map when it is possible to tolerate a decrease in the accuracy of the calculated leaf area density.

The leaf area index calculation unit 16 receives input of the leaf area density in the polarized image from the understory leaf region detection unit 142. Subsequently, the leaf area index calculation unit 16 calculates the leaf area index using the acquired leaf area density. The leaf area index calculation unit 16 outputs the calculated leaf area index to the reflection characteristic model generation unit 18.

Here, the plant characteristic extraction unit 14, the reflection characteristic estimation unit 15, the leaf area index calculation unit 16, and the reflectance calculation unit 17 correspond to an example of a “parameter calculation unit”.

The reflection characteristic model generation unit 18 receives input of the LIDF from the ellipse model fitting unit 141. In addition, the reflection characteristic model generation unit 18 receives input of the reflectance ρs of the soil from the reflectance calculation unit 17. Furthermore, the reflection characteristic model generation unit 18 receives input of the leaf area index from the leaf area index calculation unit 16.

Subsequently, by using the LIDF, the reflectance ρs of the soil, and the leaf area index, the reflection characteristic model generation unit 18 acquires the reflection characteristic model illustrated in FIG. 4 in each pixel of the polarized image. For example, the reflection characteristic model generation unit 18 determines the LIDF, the reflectance ρs of the soil, and the leaf area index in the PROSAIL model from the acquired information among the parameters of the PROSAIL model illustrated in FIG. 8. Furthermore, the value of each of parameters 215 can be obtained by actual measurement, and the reflection characteristic model generation unit 18 sets the input measured value as the value of each of the parameters 215. Furthermore, in the reflection characteristic model generation unit 18, parameters 216 are set to predetermined fixed values. Furthermore, parameters 217 are values determined by the environment of the field, and the reflection characteristic model generation unit 18 uses the input values as the values of the parameters 217. By determining individual parameters in this manner, the reflection characteristic model generation unit 18 can generate a PROSAIL model as a reflection characteristic model. The reflection characteristic model generation unit 18 outputs the generated reflection characteristic model to the vegetation index generation device 3. This reflection characteristic model generation unit 18 corresponds to an example of a “characteristic estimation unit”.

Returning to FIG. 1, the description will follow. The vegetation index generation device 3 includes an image acquisition unit 31, a correction unit 32, a vegetation index calculation unit 33, and a display control unit 34. The vegetation index generation device 3 is connected to the drone 2 by a wireless or wired channel.

The image acquisition unit 31 acquires data of a reflection spectroscopic image group from the drone 2. The image acquisition unit 31 then outputs each of reflection spectroscopic images of the acquired reflection spectroscopic image group to the correction unit 32 together with the information regarding the corresponding polarized image.

The correction unit 32 receives input of the reflection spectroscopic image group from the image acquisition unit 31. In addition, the correction unit 32 acquires the information regarding the reflection characteristic model acquired by the image processing apparatus 1. The correction unit 32 then corrects the reflection spectroscopic image by using the reflection characteristic model at each point on the reflection spectroscopic image. Subsequently, the correction unit 32 outputs the corrected reflection spectroscopic image to the vegetation index calculation unit 33.

The vegetation index calculation unit 33 receives input of the corrected reflection spectroscopic image from the correction unit 32. Subsequently, the vegetation index calculation unit 33 acquires the amount of red light and the amount of near-infrared light from the corrected reflection spectroscopic image, and calculates the VI including the NDVI. Thereafter, the corrected reflection spectroscopic image outputs the VI including the calculated NDVI to the display control unit 34. Here, although the present embodiment will describe the NDVI as an example, the information calculated from the reflection spectroscopic image corrected by the vegetation index calculation unit 33 may be another VI.

The display control unit 34 receives input of the VI including the NDVI from the vegetation index calculation unit 33. Subsequently, the display control unit 34 controls to display the VI including the NDVI on a display device such as a monitor. The user determines the vegetation status using the VI including the provided NDVI.

[Method for Generating Reflection Characteristic Model]

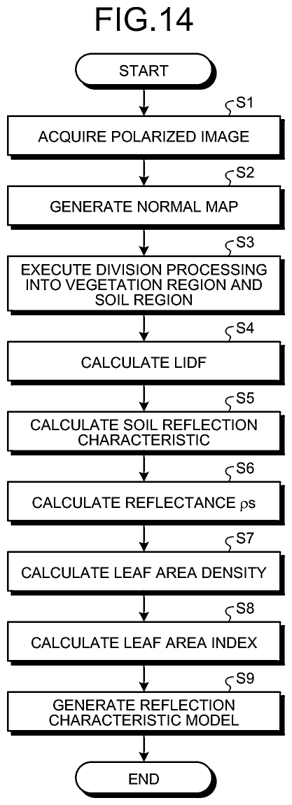

FIG. 14 is a flowchart of a reflection characteristic model generation process. Next, the flow of the reflection characteristic model generation process will be described with reference to FIG. 14.

The drone 2 captures a polarized image while flying over the field. The polarized image acquisition unit 11 acquires the polarized image of the field captured from the sky by the drone 2 (step S1).

The normal map generation unit 12 receives, from the polarized image acquisition unit 11, the input of the polarized image of the field captured from the sky. Next, the normal map generation unit 12 executes image processing on the polarized image, detects the normal of the leaf on a pixel-by-pixel basis, and generates a normal map (step S2). The normal map generation unit 12 outputs the generated normal map to the ellipse model fitting unit, the understory leaf region detection unit 142, and the reflection characteristic estimation unit 15.

The soil separation processing unit 13 receives, from the polarized image acquisition unit 11, input of the polarized image of the field captured from the sky. Next, the soil separation processing unit 13 executes the division processing into the vegetation region and the soil region on the polarized image by using a color segmentation technique (step S3). The soil separation processing unit 13 outputs the signal of the image of the vegetation region in the polarized image to the ellipse model fitting unit and the understory leaf region detection unit 142. Furthermore, the soil separation processing unit 13 outputs the signal of the image of the soil region in the polarized image to the reflection characteristic estimation unit 15.

The ellipse model fitting unit 141 receives input of the normal map from the normal map generation unit 12. Furthermore, the ellipse model fitting unit 141 receives input of the signal of the image of the vegetation region from the soil separation processing unit 13. By using the normal distribution for the information on the vegetation region in the normal map, the ellipse model fitting unit 141 obtains the optimum parameters for the mathematical approximation model using an ellipse, and calculates the LIDF (step S4). Thereafter, the ellipse model fitting unit 141 outputs the calculated LIDF to the reflection characteristic model generation unit 18.

The reflection characteristic estimation unit 15 receives input of the normal map from the normal map generation unit 12. Furthermore, the reflection characteristic estimation unit 15 receives input of the signal of the image of the soil region from the soil separation processing unit 13. Subsequently, the reflection characteristic estimation unit 15 calculates the soil reflection characteristic of the soil region using the image data and the normal map (step S5).

The reflectance calculation unit 17 calculates the reflectance ρs of the soil using the soil reflection characteristic calculated by the reflection characteristic estimation unit 15 (step S6). The reflectance calculation unit 17 outputs the calculated reflectance ρs of the soil to the reflection characteristic model generation unit 18.

The understory leaf region detection unit 142 receives input of the normal map from the normal map generation unit 12. Furthermore, the ellipse model fitting unit 141 receives input of the signal of the image of the vegetation region from the soil separation processing unit 13. Subsequently, the understory leaf region detection unit 142 obtains the number of leaves from the normal map and the signals of the image of the vegetation region by using edge detection and machine learning, and then calculates the leaf area density using the obtained number of leaves (step S7). The understory leaf region detection unit 142 outputs the calculated leaf area density to the leaf area index calculation unit 16.

The leaf area index calculation unit 16 receives an input of the leaf area density from the understory leaf region detection unit 142. Next, the leaf area index calculation unit 16 calculates the leaf area index using the leaf area density (step S8). The leaf area index calculation unit 16 outputs the calculated leaf area index to the reflection characteristic model generation unit 18.

The reflection characteristic model generation unit 18 receives input of the LIDF from the ellipse model fitting unit 141. In addition, the reflection characteristic model generation unit 18 receives input of the reflectance ρs of the soil from the reflectance calculation unit 17. Furthermore, the reflection characteristic model generation unit 18 receives input of the leaf area index from the leaf area index calculation unit 16. Subsequently, the reflection characteristic model generation unit 18 generates a reflection characteristic model using the information determined in advance, and the input information, as well as the acquired LIDF, reflectance ρs, and leaf area index (step S9).

[Action/Effects]

As described above, the image processing apparatus 1 according to the present embodiment acquires a polarized image group obtained by imaging the field from the sky. Subsequently, the image processing apparatus 1 obtains the LIDF using a normal map. Furthermore, the image processing apparatus 1 obtains the leaf area density by using the signal of the image of the vegetation region in the polarized image and using the normal map, and then calculates the leaf area index. In addition, the image processing apparatus 1 calculates the reflectance ρs of the soil by using the signal of the image of the soil region in the polarized image and using the normal map. In this manner, the image processing apparatus 1 can generate a reflection characteristic model using a wide range of information easily available in the field.

In this case, the reflection characteristic model is guaranteed to have high accuracy for the appropriate range in the field. In addition, by correcting the reflection spectroscopic image using the reflection characteristic model generated by the image processing apparatus 1 according to the present embodiment, it is possible to accurately suppress the variation due to the observation conditions. Accordingly, it is possible to implement accurate reflection spectroscopic remote sensing by using the reflection spectroscopic image corrected by using the reflection characteristic model generated by the image processing apparatus 1 according to the present embodiment.

### Second Embodiment

In contrast to the first embodiment in which the camera included in the imaging device 21 mounted on the drone 2 is simply categorized as a camera that captures a reflection spectroscopic image and a polarized image, the following will describe details of the camera included in the imaging device 21.

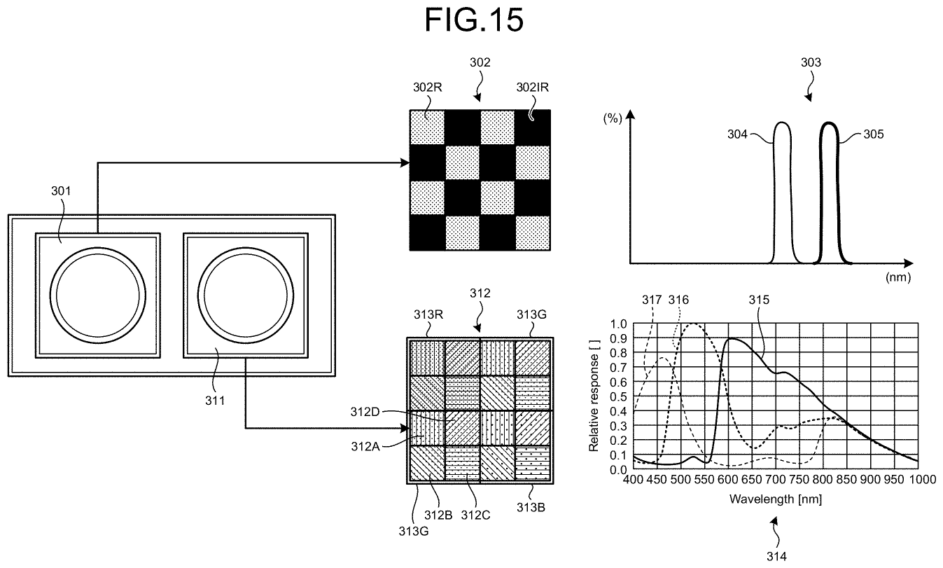

FIG. 15 is a diagram illustrating an example of an imaging device according to a second embodiment. The imaging device 21 according to the present embodiment mounted on the drone 2 performs imaging by using two cameras, namely, a camera that acquires a reflection spectroscopic image and a camera that acquires a polarized image. The details of the imaging device will be described below.

The imaging device 21 according to the present embodiment includes cameras 301 and 311. As illustrated in a pixel array 302, the camera 301 has pixels 302R each being provided with a color filter that transmits red light in the neighborhood of 650 nm in a narrowband corresponding to red. Here, the neighborhood includes a range of 50 nm on each side of the range. This narrowband in the neighborhood of 650 nm is an example of a “first predetermined narrowband”. Furthermore, the camera 301 has pixels 302IR each being provided with a color filter that transmits near-infrared light in the neighborhood of 850 nm in a narrowband corresponding to the near-infrared band. This narrowband in the neighborhood of 850 nm is an example of a “second predetermined narrowband”. The pixels 302R and pixels 302IR are alternately arranged in a checkerboard pattern. The camera 301 is a narrowband R/IR camera that simultaneously captures the red band and the near-infrared band. The camera 301 acquires a reflection spectroscopic image.

Specifically, the camera 301 acquires a signal illustrated in a graph 303. The graph 303 represents the light transmittance for each wavelength acquired by the camera 301. In the graph 303, the vertical axis represents the light transmittance and the horizontal axis represents the wavelength. A curve 304 represents the light transmittance of each frequency band acquired by the pixel 302R, and corresponds to the transmittance of red light. Furthermore, a curve 305 represents the light transmittance for each wavelength acquired by the pixel 302IR, and corresponds to the transmittance of near-infrared light. The correction unit 32 of the vegetation index generation device 3 can acquire the NDVI, which is a vegetation index, from the reflection spectroscopic image captured by the camera 301. This camera 301 corresponds to an example of a “first camera”.

The camera 311 is a polarization camera that acquires a polarized image. As illustrated in the pixel array 312, the camera 311 includes arrangements of the following three color filters. One is a color filter 313R (hereinafter referred to as a “red filter”) that selectively transmits light having a red wavelength component. Another one is a color filter 313G (hereinafter referred to as a “green filter”) that selectively transmits light having a green wavelength component. The other one is a color filter 313B (hereinafter referred to as a “blue filter”) that selectively transmits light having a blue wavelength component. In addition, the camera 311 includes polarized lenses 312A, 312B, 313C and 312D of four angles, namely, 0 degrees, 45 degrees, 90 degrees and 135 degrees, for each of the color filters. In the following, polarization signals having four angles of 0 degrees, 45 degrees, 90 degrees, and 135 degrees are referred to as 4-direction polarization signals. That is, the camera 311 has three channels for the color and each of the three color channels has four channels for acquiring 4-direction polarization signals, making it possible to acquire an image of signals having a total of 12 channels. In the present embodiment, the camera 311 has the green filters 313G arranged twice as much as the images of the red filters 313R and the blue filters 313B. However, the distribution of this color filters may be arranged in different distribution.

A graph 314 illustrates the relative response of red, green, and blue for each wavelength when captured by the camera 311. In graph 314, the vertical axis represents the relative response and the horizontal axis represents the wavelength. A curve 315 represents the response of red. A curve 316 represents the response of green. A curve 317 represents the response of blue. This camera 311 is an example of a “second camera”.

The image processing apparatus 1 obtains the LIDF, the leaf area index, and the reflectance ρs of the soil from the polarized image formed by the light represented in the graph 313 acquired by the camera 311 and then generates a reflection characteristic model. By correcting the NDVI generated from the graph 303 acquired by the camera 301 by using the generated reflection characteristic model, the user of the image processing apparatus 1 can implement accurate reflection spectroscopic remote sensing.

As described above, by using a camera having pixels including arrangements of a color filter that transmits light in the neighborhood of 650 nm in a narrowband and a color filter that transmits light in the neighborhood of 850 nm in a narrowband, it is possible to acquire a reflection spectroscopic image in a red band and a gold infrared light band. Furthermore, a polarized image can be acquired by performing imaging using a camera including pixels of four polarization directions assigned to each of three colors. Additionally, by using the polarized image, the image processing apparatus 1 can acquire each of parameters used for correction. With this configuration, the image processing system 100 can appropriately correct the NDVI and can generate an accurate reflection spectroscopic image. By using the accurate reflection spectroscopic image, the user of the image processing system can implement accurate reflection spectroscopic remote sensing.

(Modifications)

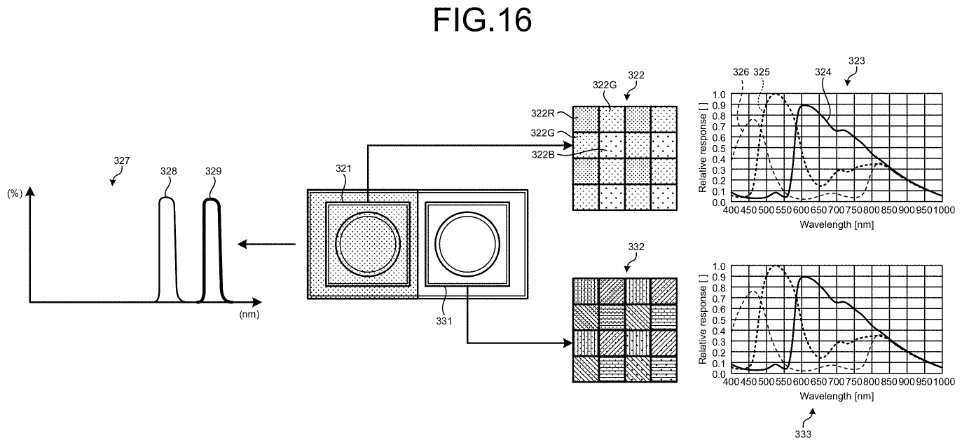

FIG. 16 is a diagram illustrating an example of an imaging device according to a modification of the second embodiment. The imaging device 21 according to the present modification is different from the second embodiment regarding the narrowband R/IR camera in that a bandpass filter that transmits two wavelength bands is disposed directly above or below the lens and that a normal RGB filter is used as a color filter on each of the pixels.

The imaging device 21 according to the present modification includes cameras 321 and 331 illustrated in FIG. 16. In the camera 321, as illustrated by a pixel array 322, a red color filter is disposed on a pixel 322R, a green color filter is disposed on a pixel 322G, and a blue color filter is disposed on a pixel 322B. Combinations of four pixels including the pixels 322R, 322G and 322B are repeatedly arranged on the camera 321. Furthermore, the lens of the camera 321 is provided with a bandpass filter that passes two wavelength bands, one in the neighborhood of 650 nm in the narrowband corresponding to red and the other in the neighborhood of 850 nm in the narrowband corresponding to the near-infrared band.

In the camera 321, with the passage of light through the RGB filter, red, blue, and green are acquired with the relative transmittance for each wavelength illustrated in a graph 323. A curve 324 represents the relative transmittance of red, a curve 335 represents the relative transmittance of green, and a curve 336 represents the relative transmittance of blue. Furthermore, with the passage of light through the bandpass filter that passes two wavelength bands, the camera 321 acquires the light in the bands illustrated in a graph 327. In the graph 327, the vertical axis represents the transmittance and the horizontal axis represents the wavelength. That is, the camera 321 acquires the narrowband light in the neighborhood of 650 nm illustrated by a curve 328 and the narrowband light in the neighborhood of 850 nm illustrated by a curve 329.

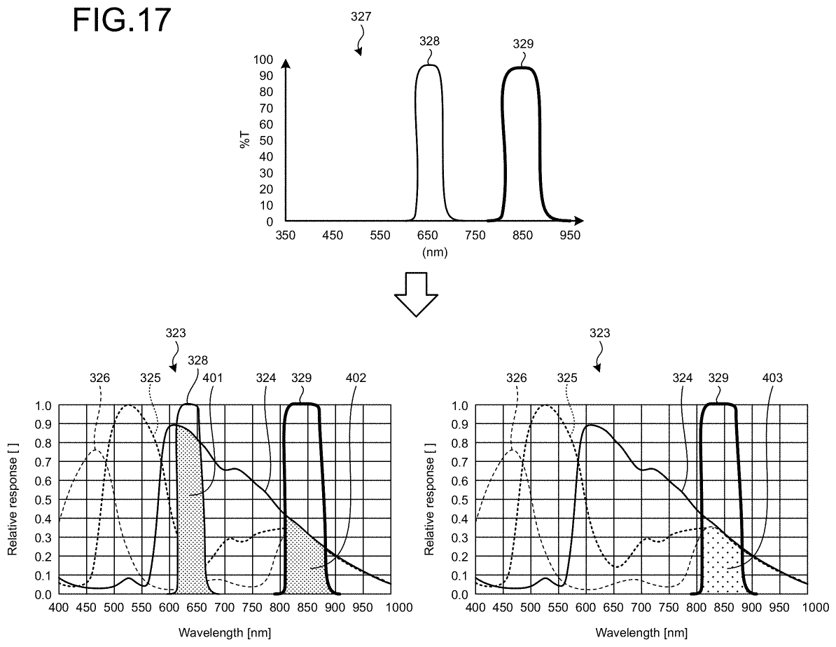

FIG. 17 is a diagram illustrating acquisition of a narrowband R/IR signal by a combination of a bandpass filter and an RGB sensor. The bandpass filter of the camera 321 passes light in the wavelength range illustrated by curves 328 and 329 illustrated in FIG. 17. In the graph 323, the integrated relative response is the amount of light acquired by the camera 321. Therefore, with respect to the red color among the beams of light illustrated in the graph 323, the camera 321 acquires the light corresponding to regions 401 and 402, which are portions in which the curve 324 overlap with the curves 328 and 329. In addition, with respect to the blue color among the beams of light illustrated in the graph 323, the camera 321 acquires the light corresponding to a region 403, which is a portion in which the curve 326 overlap with the curves 328 and 329. The near-infrared light is represented as light corresponding to the region 403. The red light is obtained as a value by first multiplying the total amount of transmitted red light by a weight, the weight obtained by dividing the region 403 by the region 402, and then dividing the multiplication result by the total amount of the blue light dropped. That is, “the amount of near-infrared light=the amount of light corresponding to the region 403”. Furthermore, “the amount of near-infrared light=total amount of transmitted red light×weight−total amount of transmitted blue light”.

In this manner, by combining the bandpass filter with the RGB filter, the camera 321 can acquire narrowband red light in the neighborhood of 650 nm and narrowband near-infrared light in the neighborhood of 850 nm. Since it is difficult to manufacture the narrowband color filter arranged above the pixels described in the second embodiment, using the camera 321 having the combination of the bandpass filter and the RGB filter described in the modification will further facilitate manufacture of the imaging device 21.

The camera 331 has a configuration similar to the camera 311 in the second embodiment. A pixel array 332 of each pixel in the camera 311 is similar to the pixel array 312 of FIG. 15. The light captured by the camera 331 is represented by a graph 333.

In this case as well, the image processing apparatus 1 obtains the LIDF, the leaf area index, and the reflectance ρs of the soil from the image captured by the camera 331, and generates a reflection characteristic model. By correcting the NDVI acquired by the camera 321 by using the generated reflection characteristic model, the user of the image processing apparatus 1 can implement accurate reflection spectroscopic remote sensing.

As described above, by using a camera combining a bandpass filter and an RGB filter, it is possible to acquire a reflection spectroscopic image of a red band and a gold infrared light band. In this manner, even with a camera that combines a bandpass filter and an RGB filter, it is possible to acquire a reflection spectroscopic image, making it possible for the image processing system 100 to appropriately correct the NDVI and generate an accurate reflection spectroscopic image. By using the accurate reflection spectroscopic image, the user of the image processing system can implement accurate reflection spectroscopic remote sensing.

### Third Embodiment

The second embodiment has described a case where the drone 2 is equipped with the imaging device 21 including two cameras. In contrast, the drone 2 according to the present embodiment is equipped with an imaging device 21 including three cameras, each of which captures normal RGB signals, narrowband R/IR signals, and 4-direction polarization signals, respectively. Details of the cameras included in the imaging device 21 mounted on the drone 2 according to the present embodiment will be described below.

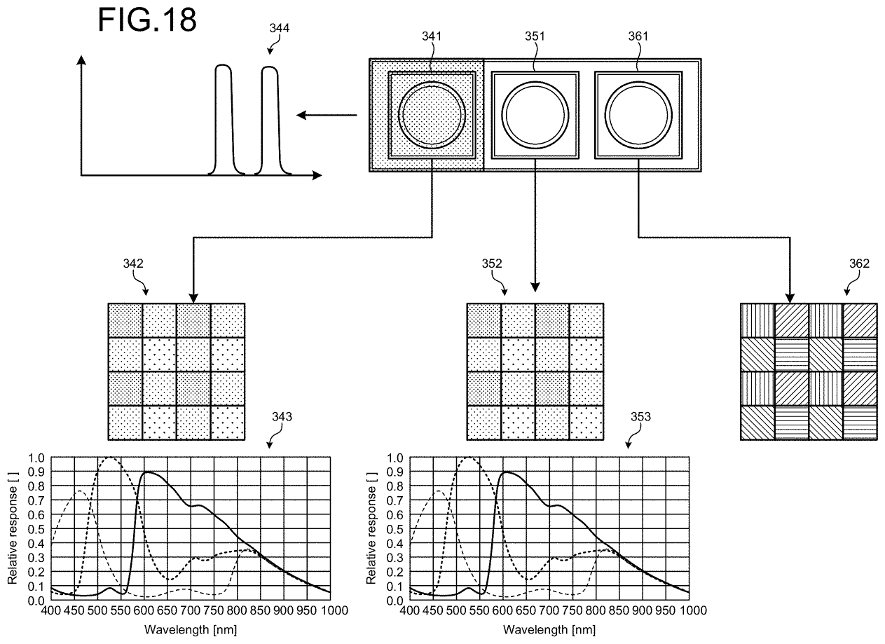

FIG. 18 is a diagram illustrating an example of an imaging device according to a third embodiment. The imaging device 21 according to the present embodiment includes cameras 341, 351 and 361.

The camera 341 is a camera that acquires narrowband red light in the neighborhood of 650 nm and narrowband near-infrared light in the neighborhood of 850 nm by combining a bandpass filter and an RGB filter. The camera 341 has the function similar to the camera 321 according to the modification of the second embodiment illustrated in FIG. 16. Specifically, the camera 341 has a bandpass filter that transmits light in the wavelength range illustrated in a graph 344, provided above the lens. Furthermore, the camera 341 has an RGB filter above each pixel in the pattern illustrated by a pixel array 342, and acquires the light in the wavelength range illustrated in the graph 344 from among the light beams represented by a graph 343 to generate an image. This camera 341 is an example of a “first camera”.

The camera 351 has an RGB filter arranged above each pixel in a pattern represented by a pixel array 352. The camera 351 acquires the light represented by a graph 353 and generates a normal RGB image. This camera 351 is an example of a “second camera”.

The camera 361 has pixels each being equipped with a black-and-white sensor and configured to acquire polarization signals in four directions as illustrated by a pixel array 362. That is, the camera 361 generates a black-and-white polarized image using polarization signals in four directions. This camera 361 is an example of a “third camera”.

The image processing apparatus 1 creates a normal map using the normal RGB image acquired by the camera 351 and the polarized image acquired by the camera 361, and calculates the LIDF, the reflectance ρs, and the leaf area index.

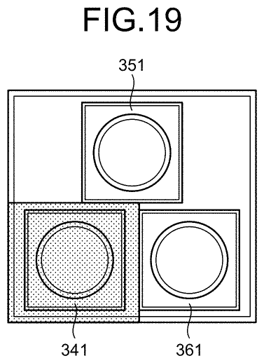

Here, the above has described the case where three cameras are arranged in a row. However, the arrangement of cameras is not limited to this. FIG. 19 is a diagram illustrating another example of the imaging device according to the third embodiment. For example, as illustrated in FIG. 19, as camera arrangement, the camera 341 and the camera 361 may be arranged side by side, and another camera, namely the camera 351 may be arranged on the side in a line-up direction. That is, the cameras 341, 351 and 361 may be arranged so as to form a triangle.

As described above, the LIDF, the reflectance ρs, and the leaf area index are calculated by using images captured by the cameras that capture the normal RGB signal and the 4-direction polarization signals and that is included in the imaging device 21 mounted on the drone 2 according to the present embodiment. In this manner, by decolorizing the polarization signal, it is possible to increase the spatial resolution and the amount of light of each channel.

### Fourth Embodiment

The imaging device 21 mounted on the drone 2 according to the present embodiment uses each pixel as a black-and-white sensor in all cameras and acquires each signal with a filter. Details of the cameras included in the imaging device 21 mounted on the drone 2 according to the present embodiment will be described below.

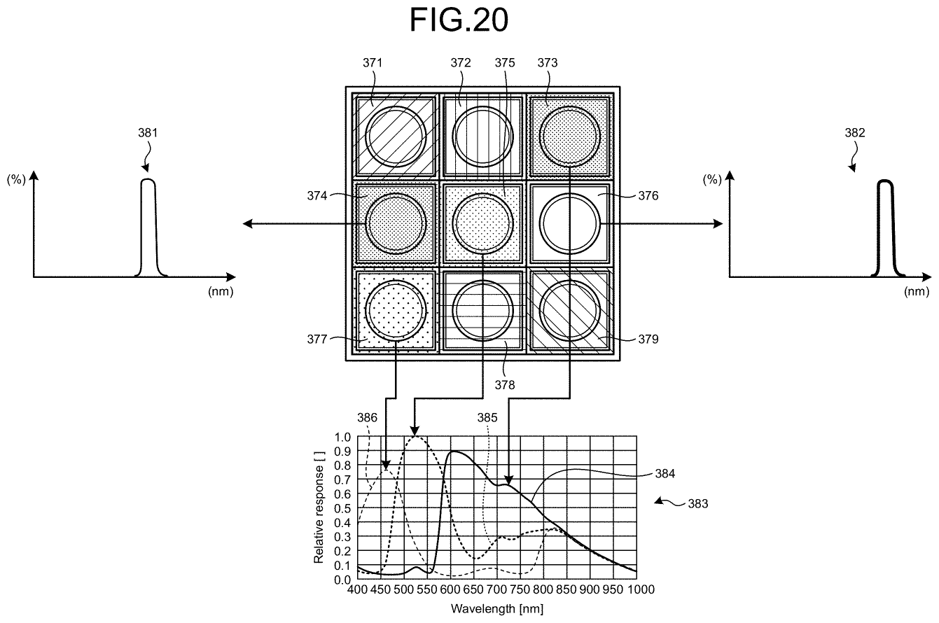

FIG. 20 is a diagram illustrating an example of an imaging device according to a fourth embodiment. As illustrated in FIG. 20, the imaging device 21 mounted on the drone 2 according to the present embodiment has nine cameras, namely, cameras 371 to 379.

Each of the cameras 371 to 379 has a black-and-white sensor. In the cameras 371, 372, 378 and 379, filters for transmitting polarization signals in four mutually different directions are arranged directly above or directly below the lenses. With this configuration, the cameras 371 to 379 generate a polarized image captured by the 4-direction polarization signals.

The camera 373 has a red color filter arranged directly above or directly below the lens. The camera 375 has a green color filter arranged directly above or directly below the lens. The camera 377 has a blue color filter arranged directly above or directly below the lens. The camera 373 acquires the red light represented by a curve 384 in a graph 383. The camera 375 acquires the green light represented by a curve 385 in the graph 383. The camera 377 acquires the blue light represented by a curve 386 in the graph 383. That is, a normal RGB image is generated by the cameras 373, 357 and 377.

The camera 374 includes a bandpass filter that allows passage of light in a narrowband in the neighborhood of 650 nm corresponding to red arranged directly above or directly below the lens. The camera 374 acquires light that has passed through the wavelength band illustrated in a graph 382.

The camera 376 includes a bandpass filter that allows passage of light in a narrowband in the neighborhood of 850 nm corresponding to blue arranged directly above or directly below the lens. The camera 376 acquires light that has passed through the wavelength band illustrated in a graph 382.

The image processing apparatus 1 calculates LIDF, reflectance ρs, and leaf area index using normal RGB images generated by the cameras 373, 375, and 377 and polarized images generated by the cameras 371, 372, 378, and 379, and generates a reflection characteristic model.

The image processing apparatus 1 in the image processing system 100 acquires NDVI using the normal RGB image generated by the cameras 373, 375 and 377, and the narrowband signal in the neighborhood of 650 nm and the narrowband signal in the neighborhood of 850 nm acquired by the cameras 374 and 375. Furthermore, the image processing apparatus 1 corrects the acquired NDVI using the reflection characteristic model generated by the image processing apparatus 1.

As described above, the imaging device 21 mounted on the drone 2 according to the present embodiment includes a camera in which an RGB color filter, a 4-direction polarizing filter, or a bandpass filter is arranged together with a black-and-white sensor. Even with such a configuration, the image processing apparatus 1 can acquire a reflection spectroscopic image and a polarized image, and can accurately obtain a reflection characteristic model. Furthermore, the imaging device 21 according to the present embodiment can increase the spatial resolution and the amount of light of each channel, similarly to the imaging device 21 according to the third embodiment.

Here, although each of the above embodiments has described a configuration in which different polarizing filters are arranged, the configuration is not limited to this, and the imaging device 21 mounted on the drone 2 may acquire 4-direction polarization signals by changing the polarization angle of a single polarization sensor. In that case, for example, the imaging device 21 in the fourth embodiment will have six cameras.

Furthermore, although each of the above embodiments is an example of generating a polarized image using polarization signals in four directions, the normal map using the polarized image in practice can be generated by a polarized image using polarization signals in at least three directions. Still, by increasing the direction of the polarization signal, the accuracy of normal on the normal map can be improved. For example, in the case of the imaging device 21 of the fourth embodiment, the configuration of a camera that acquires three or more different polarization direction signals can be considered.

Furthermore, each of the above embodiments has described a case of recording the reflection spectroscopic image and the dimmed image from the camera mounted on the drone 2. However, the polarized image may be captured by a camera near the surface of the earth, separately from the camera mounted on the drone 2. For example, in the case of reflection spectroscopic analysis based on a satellite image, it is preferable to capture a polarized image with a camera near the surface of the earth.

Furthermore, even when the drone 2 is equipped with a camera for capturing a polarized image, it is allowable to capture a polarized image separately from the capture of the reflection spectroscopic image. For example, before capturing the reflection spectroscopic image, the polarized image may be captured at an altitude different from the altitude in the capture of the reflection spectroscopic image. Specifically, a polarized image of a part of the region of the field may be captured in advance at a lower altitude. Furthermore, the polarized image may be captured by using a drone 2 different from the drone 2 that captures the reflection spectroscopic image.

Furthermore, although each of the above embodiments has described the case of using an image in which both the vegetation region and the soil region are recorded, the image used by the image processing apparatus 1 is not limited to this. For example, the image processing apparatus 1 may use a vegetation region and a soil region recorded as separate images, and may obtain the LIDF and leaf area index based on the image of the vegetation region and obtain the reflectance ρs of the soil based on the image of the soil region. In this case, the image processing apparatus 1 does not have to include the soil separation processing unit 13.

The embodiments of the present disclosure have been described above. However, the technical scope of the present disclosure is not limited to the above-described embodiments, and various modifications can be made without departing from the scope of the present disclosure. Moreover, it is allowable to combine the components across different embodiments and a modification as appropriate.

The effects described in the present specification are merely examples, and thus, there may be other effects, not limited to the exemplified effects.

Note that the present technology can also have the following configurations.

(1)

An image processing apparatus comprising:

a vector analysis unit that obtains a normal vector characteristic based on a polarized image acquired; and

a characteristic estimation unit that estimates a reflection characteristic model based on the normal vector characteristic obtained by the vector analysis unit.

(2)

The image processing apparatus according to (1), further comprising

a parameter calculation unit that calculates parameters included in the reflection characteristic model based on the normal vector characteristic,

wherein the characteristic estimation unit estimates the reflection characteristic model using the parameters calculated by the parameter calculation unit.

(3)

The image processing apparatus according to (2), wherein the parameter calculation unit calculates a parameter representing normal distribution as the parameter.

(4)

The image processing apparatus according to (3), wherein the parameter calculation unit calculates the parameter representing the normal distribution using an ellipse model.

(5)

The image processing apparatus according to any one of (2) to (4), wherein the parameter calculation unit calculates a parameter representing reflectance of soil as the parameter.

(6)

The image processing apparatus according to any one of (2) to (5), wherein the parameter calculation unit calculates a parameter representing a leaf area index, which is a leaf occupancy ratio within a unit area, as the parameter.

(7)

The image processing apparatus according to any one of (1) to (6), wherein the vector analysis unit acquires the polarized image from an imaging device having a first camera that captures a reflection spectroscopic image and a second camera that captures the polarized image.

(8)

The image processing apparatus according to (7),

wherein the first camera includes a pixel array in which pixels in which a filter that transmits red light in a first predetermined narrowband is disposed and pixels in which a filter that transmits near-infrared light in a second predetermined narrowband is disposed are alternately arranged, and

the second camera includes a pixel that acquires polarization signals in at least three directions for each of red light, green light, and blue light.

(9)

The image processing apparatus according to (7),

wherein the first camera includes a pixel in which a first filter that transmits one of red light, green light, or blue light is disposed and in which a filter that transmits one of a wavelength of a first predetermined narrowband or a wavelength of a second predetermined narrowband is disposed to be superimposed with the first filter, and

the second camera includes a pixel that acquires polarization signals in at least three directions for each of red light, green light, and blue light.

(10)

The image processing apparatus according to any one of (1) to (6), wherein, from an imaging device including: a first camera that captures a reflection spectroscopic image; a second camera that captures a color image; and a third camera that captures a black-and-white polarized image, the vector analysis unit acquires the color image and the black-and-white polarized image so as to be applied as the polarized image.

(11)

The image processing apparatus according to any one of (1) to (6), wherein the vector analysis unit acquires a polarized image from an imaging device equipped with a camera including a white-and-black sensor equipped with any one of: a filter that transmits red light in a first predetermined narrowband; a filter that transmits infrared light in a second predetermined narrowband; a filter that passes red light; a filter that passes green light; a filter that passes blue light; and a changing filter that passes a biased component.

(12)

An image processing method comprising:

obtaining a normal vector characteristic based on a polarized image acquired; and

estimating a reflection characteristic model based on the normal vector characteristic.

(13)

An image processing program causing a computer to execute processes comprising:

obtaining a normal vector characteristic based on a polarized image acquired; and

estimating a reflection characteristic model based on the normal vector characteristic.

### REFERENCE SIGNS LIST

1 IMAGE PROCESSING APPARATUS
2 DRONE
3 VEGETATION INDEX GENERATION DEVICE
11 POLARIZED IMAGE ACQUISITION UNIT
12 NORMAL MAP GENERATION UNIT
13 SOIL SEPARATION PROCESSING UNIT
14 PLANT CHARACTERISTIC EXTRACTION UNIT
15 REFLECTION CHARACTERISTIC ESTIMATION UNIT
16 LEAF AREA INDEX CALCULATION UNIT
17 REFLECTANCE CALCULATION UNIT
18 REFLECTION CHARACTERISTIC MODEL GENERATION UNIT
21 IMAGING DEVICE
31 IMAGE ACQUISITION UNIT
32 CORRECTION UNIT
33 VEGETATION INDEX CALCULATION UNIT
34 DISPLAY CONTROL UNIT

1. An image processing apparatus comprising:
a vector analysis unit that obtains a normal vector characteristic based on a polarized image acquired; and
a characteristic estimation unit that estimates a reflection characteristic model based on the normal vector characteristic obtained by the vector analysis unit.

2. The image processing apparatus according to claim 1, further comprising
a parameter calculation unit that calculates parameters included in the reflection characteristic model based on the normal vector characteristic,
wherein the characteristic estimation unit estimates the reflection characteristic model using the parameters calculated by the parameter calculation unit.

3. The image processing apparatus according to claim 2, wherein the parameter calculation unit calculates a parameter representing normal distribution as the parameter.

4. The image processing apparatus according to claim 3, wherein the parameter calculation unit calculates the parameter representing the normal distribution using an ellipse model.

5. The image processing apparatus according to claim 2, wherein the parameter calculation unit calculates a parameter representing reflectance of soil as the parameter.

6. The image processing apparatus according to claim 2, wherein the parameter calculation unit calculates a parameter representing a leaf area index, which is a leaf occupancy ratio within a unit area, as the parameter.

7. The image processing apparatus according to claim 1, wherein the vector analysis unit acquires the polarized image from an imaging device having a first camera that captures a reflection spectroscopic image and a second camera that captures the polarized image.

8. The image processing apparatus according to claim 7,
wherein the first camera includes a pixel array in which pixels in which a filter that transmits red light in a first predetermined narrowband is disposed and pixels in which a filter that transmits near-infrared light in a second predetermined narrowband is disposed are alternately arranged, and
the second camera includes a pixel that acquires polarization signals in at least three directions for each of red light, green light, and blue light.

9. The image processing apparatus according to claim 7,
wherein the first camera includes a pixel in which a first filter that transmits one of red light, green light, or blue light is disposed and in which a filter that transmits one of a wavelength of a first predetermined narrowband or a wavelength of a second predetermined narrowband is disposed to be superimposed with the first filter, and
the second camera includes a pixel that acquires polarization signals in at least three directions for each of red light, green light, and blue light.

10. The image processing apparatus according to claim 1, wherein, from an imaging device including: a first camera that captures a reflection spectroscopic image; a second camera that captures a color image; and a third camera that captures a black-and-white polarized image, the vector analysis unit acquires the color image and the black-and-white polarized image so as to be applied as the polarized image.

11. The image processing apparatus according to claim 1, wherein the vector analysis unit acquires a polarized image from an imaging device equipped with a camera including a white-and-black sensor equipped with any one of: a filter that transmits red light in a first predetermined narrowband; a filter that transmits infrared light in a second predetermined narrowband; a filter that passes red light; a filter that passes green light; a filter that passes blue light; and a changing filter that passes a biased component.

12. An image processing method comprising:
obtaining a normal vector characteristic based on a polarized image acquired; and
estimating a reflection characteristic model based on the normal vector characteristic.

13. An image processing program causing a computer to execute processes comprising:
obtaining a normal vector characteristic based on a polarized image acquired; and
estimating a reflection characteristic model based on the normal vector characteristic.
